(19) United States
(12) Patent Application Publication
(10) Pub. No.: US 2023/0414934 A1
(43) Pub. Date: Dec. 28, 2023

(54) SURGICAL INSTRUMENT

(71) Applicant: Neurosurgery & Spine Medical Expert LLC, Eureka, MO (US)

(72) Inventor: Tobias Alecio Mattei, Eureka, MO (US)

(21) Appl. No.: 18/212,625

(22) Filed: Jun. 21, 2023

Related U.S. Application Data
(60) Provisional application No. 63/354,561, filed on Jun. 22, 2022.

(51) Int. Cl. A61N 1/05; A61B 34/30

(52) U.S. Cl. CPC A61N 1/0553; A61B 2034/306; A61N 1/0558; A61B 34/30

(57) ABSTRACT

A surgical instrument for positioning a paddle electrode. The instrument includes a pair of flexibly resilient arms operatively coupled at their user ends. Each arm extends along a rearward section from its user end to an intermediate section extending obliquely upward to a forward section extending obliquely downward relative to the intermediate section. The instrument includes a jaw formed adjacent to a working end of each arm and a guide located along the forward section of each arm. The jaws are separated by a gap that is larger than a distance between the side edges of the electrode when the arms are unflexed. The arms are capable of flexing sufficiently to engage the jaws with the side edges of the paddle electrode and sufficiently resilient to return to a released configuration in which the gap is larger than the distance between the opposite side edges of the electrode.

### CROSS-REFERENCE TO RELATED APPLICATIONS

The present application claims priority of co-pending U.S. Provisional Application No. 63/354,561 filed Jun. 22, 2022, and entitled, “Specialized Bayonet Forceps,” which is hereby incorporated by reference.

### BACKGROUND OF THE INVENTION

The present disclosure is directed to a surgical instrument, and more particularly, to forceps especially adapted for positioning paddle electrodes in a patient's spine.

Spinal stimulation (also called dorsal column stimulation) is procedure in which specific nerves in a patient's spine are stimulated to alter the targeted nerve activity. Although the targeted nerves may be stimulated using pharmaceutical agents, the current focus of the device and procedures discussed in this disclosure is electrically stimulating nerves in a target area of a patient's spine to treat chronic pain conditions, such as failed back syndrome, chronic radiculopathy, complex regional pain syndrome, and peripheral and diabetic polyneuropathy. Numerous trials have been conducted to explore the efficacy of spinal stimulation systems, and use of these systems is expected to increase markedly.

Practitioners currently insert electrodes of spinal stimulation systems by percutaneously placing wire electrodes or by placing paddle electrodes via open laminectomy. Using percutaneous placement, wire electrodes are inserted through the patient's skin to the target area of the spine. Paddle electrodes are positioned in the target area by surgically removing portions (i.e., laminae) of the patient's spine to expose the target area (i.e., perform an open laminectomy). First-generation spinal stimulation systems were primarily implanted percutaneously (i.e., through the skin). However, percutaneously implanting wire electrodes has inherent disadvantages, including difficulty controlling lateral movement of the electrode as it is implanted. Another disadvantage of percutaneously implanted electrodes is that the electrodes are susceptible to moving after being implanted. Further, wire electrodes inherently stimulate smaller areas than paddle electrodes potentially failing to stimulate portions of the target area resulting in suboptimal outcomes.

To eliminate the disadvantages of percutaneously implanted wire electrodes, an increasing number of practitioners are performing a thoracic laminectomy (i.e., removing a selected portion of the patient's thoracic vertebrae) to expose a selected area of a patient's spinal cord before positioning a multi-column paddle electrode adjacent to target area. Because paddle electrodes stimulate larger areas than wire electrodes, paddle electrodes are more likely to stimulate an area sufficient to encompass the entire target area.

Smaller incisions and shorter surgical times tend to reduce post-operative pain, recovery time, and potential for complications. To minimize soft tissue dissection and reduce surgical time, most practitioners perform the smallest laminectomy required to slide the paddle electrode to the selected vertebral lamina. Preserving spinous processes (i.e., protrusions extending from the vertebra) and laminae cranial to the electrode insertion site (i.e., vertebral layers adjacent to the margin of the incision that is closer to the patient's head) reduces the potential that the electrode will dislocate dorsally (i.e., move rearward) compared to performing a full laminectomy. Because no procedure-specific instruments exist for holding the paddle electrode when guiding the electrode into position, many practitioners currently use conventional bayonet forceps to grasp the electrode by the sides or trailing margin. Unfortunately, conventional bayonet forceps are not well suited for firmly gripping the paddle, frequently allowing the paddle electrode to twist during insertion. Practitioners often find it challenging to precisely direct the paddle electrode using conventional bayonet forceps. Some paddle electrodes are longer than the desirable incision length. Maneuvering the paddle electrode into position frequently requires practitioners to flex the leading portion of the paddle electrode to slide the paddle electrode through the laminectomy opening and direct it into position.

In view of the foregoing, there remains a need for an instrument that are specifically adapted to inserting spinal stimulation paddle electrodes to overcome the disadvantages found in conventional bayonet forceps. In particular, there remains a need for an instrument that enables the practitioner to firmly grip a paddle electrode by its lateral edges and guide wire leads when flexing the paddle electrode, so the size and shape of the inserted paddle electrode matches those of a minimally invasive incision.

### SUMMARY

In one aspect, the present disclosure includes a surgical instrument for positioning a paddle electrode having opposite side edges extending between a leading edge and a trailing edge opposite the leading edge. The paddle electrode has leads extending from the trailing edge. The instrument comprises a pair of flexibly resilient arms operatively coupled at a user end of each arm. Each arm extends longitudinally along a rearward section from the corresponding user end to an intermediate section extending obliquely upward relative to the rearward section to a forward section extending obliquely downward relative to the intermediate section toward a working end opposite the corresponding user end. Each arm has a cooperative face facing the other arm of the pair and an outer face facing opposite the cooperative face. In addition, the instrument includes a jaw formed adjacent to the working end of each arm adapted to engage a selected side edge of the paddle electrode when gripping the paddle electrode. The instrument also comprises a guide located along the forward section of each arm adapted to receive a selected lead of the leads of the paddle electrode when the jaws are gripping the paddle electrode. The jaws of the pair of arms are separated by a gap that is larger than a distance between the opposite side edges of the paddle electrode when the pair of arms is unflexed. The pair of arms is capable of being flexed to reduce the gap separating the jaws sufficiently to engage the jaws with the side edges of the paddle electrode to grip the paddle electrode. The pair of arms is sufficiently resilient to return the arms to a released configuration in which the gap separating the jaws is larger than the distance between the opposite side edges of the paddle electrode.

In another aspect, the present disclosure includes a surgical instrument for positioning a paddle electrode having opposite side edges extending between a leading edge and a trailing edge opposite the leading edge. The paddle electrode has leads extending from the trailing edge. The instrument comprises a pair of flexibly resilient arms operatively coupled at a user end of each arm. Each arm extends to a working end opposite the corresponding user end. Each arm has a cooperative face facing the other arm of the pair and an outer face facing opposite the cooperative face. The instrument also includes a jaw formed at and offset from the working end of each arm. Each jaw has a clamping face facing the jaw of the other arm and a groove extending across the clamping face. The width of the groove is adapted to receive a paddle electrode side edge. In addition, the instrument comprises a guide located along each arm adapted to receive a selected lead of the paddle electrode when the jaws of the arms are gripping the paddle electrode. The jaws of the pair of arms are separated by a gap that is larger than a distance between the opposite side edges of the paddle electrode when the pair of arms is unflexed. The pair of arms is capable of being flexed to reduce the gap separating the jaws sufficiently to engage the jaws with the side edges of the paddle electrode to grip the paddle electrode. The pair of arms is sufficiently resilient to return the arms to a released configuration in which the gap separating the jaws is larger than the distance between the opposite side edges of the paddle electrode.

In yet another aspect, the present disclosure includes a surgical instrument for positioning a paddle electrode having opposite side edges extending between a leading edge and a trailing edge opposite the leading edge. The paddle electrode has leads extending from the trailing edge. The instrument comprises a pair of flexibly resilient arms operatively coupled at a user end of each arm. Each arm of the pair extends to a working end opposite the corresponding user end. Each arm has a cooperative face facing the other arm of the pair and an outer face facing opposite the cooperative face. The instrument also includes a jaw formed at and offset from the working end of each arm. Each the jaw has a clamping face facing the jaw of the other arm. Further, the instrument includes a guide comprising a hollow cylindrical tube mounted on each arm adjacent to the working end of the corresponding arm. The hollow cylindrical tube extends along a centerline of the corresponding arm and has an interior diameter sized to receive at least one lead of the paddle electrode leads. The jaws of the pair of arms are separated by a gap that is larger than a distance between the opposite side edges of the paddle electrode when the pair of arms is unflexed. The pair of arms is capable of being flexed to reduce the gap separating the jaws sufficiently to engage the jaws with the side edges of the paddle electrode to grip the paddle electrode. The pair of arms is sufficiently resilient to return the arms to a released configuration in which the gap separating the jaws is larger than the distance between the opposite side edges of the paddle electrode.

Other aspects of the present disclosure will be apparent in view of the following description and claims.

### BRIEF DESCRIPTION OF THE DRAWINGS

FIG. 1 is a perspective of an example of an instrument for guiding a paddle electrode during laminectomy positioning;

FIG. 2 is a top plan of the instrument; and

FIG. 3 is a front elevation of the instrument.

Corresponding reference characters indicate corresponding parts throughout the drawings.

### DETAILED DESCRIPTION

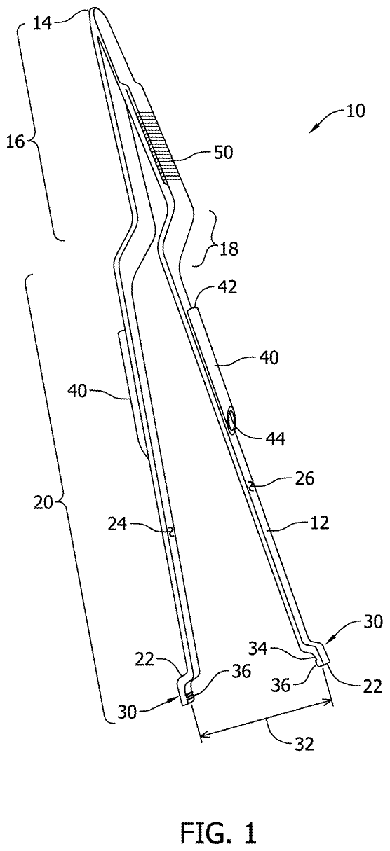

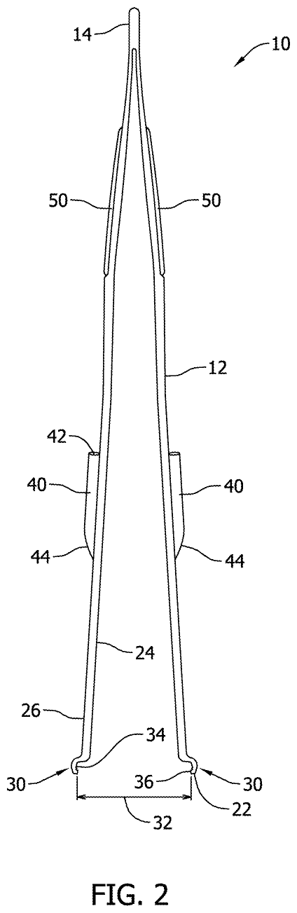

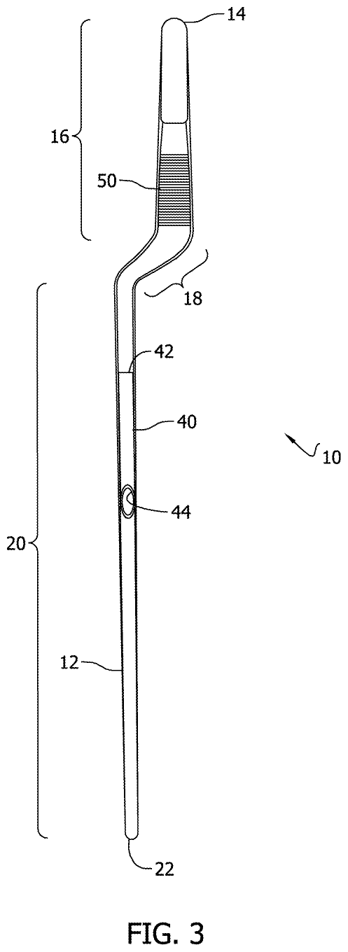

As illustrated in FIGS. 1-3, specialized bayonet forceps (broadly, a surgical instrument) for positioning spinal stimulation paddle electrodes are designated in their entirety by the reference number 10. Similar to conventional bayonet forceps, the specialized bayonet forceps 10 include a pair of opposing flexibly resilient arms 12 that are operatively coupled or joined at their respective user ends 14. Each arm 12 is bayonet shaped such that the arm extends longitudinally from the user end 14 along a rearward section 16 to an intermediate section 18 extending obliquely upward relative to the rearward section to a forward section 20 extending obliquely downward relative to the intermediate section toward a working end or tip 22 opposite the corresponding user end. Further, each arm 12 has a cooperative face 24 facing inward toward the opposite arm and an outer face 26 facing outward opposite the cooperative face.

Each arm 12 has a jaw, generally designated by 30, formed adjacent to its corresponding working end 22. Each jaw 30 is laterally offset from the working end 22 of the corresponding arm 12 as shown in FIGS. 1 & 2 such that a gap distance 32 measured between opposing clamping faces 34 of the jaws is wider than the space between the arms. A series of parallel grooves 36 is formed on the clamping face 34 of each jaw 30 to ensure the instrument 10 reliably holds a paddle electrode (not shown), particularly as the paddle electrode flexes during insertion. Each of the grooves 36 is sized and shaped to correspond to receive opposite edges of a paddle electrode. More particularly, each of the grooves 36 has a width adapted to engage or receive the side edges of the paddle electrode. Although the grooves may have other widths, the grooves 36 of the illustrated example each has a groove width is in a width range of about 3 mm to about 4 mm to match an edge thickness of most conventional paddle electrodes. In the illustrated example, two or three parallel grooves 36 are provided on the opposing inner faces 34 of the jaws 30 but it is envisioned the faces may have fewer or more grooves.

As illustrated in FIGS. 1 & 2, a guide 40 is located on the outer face 26 of each arm 12 along the corresponding forward section 20 to receive a selected lead (not shown) of a paddle electrode clamped between the jaws 30. In the illustrated example, each guide 40 consists of a hollow cylindrical tube. Although the tube may have other minimum interior widths of diameters, the illustrated tube has a diameter of about 3 mm to easily thread a conventional paddle electrode lead. In the illustrated example, each tube has a length measured along its centerline of about 25 mm and the tube is oriented with the centerline extending parallel to the forward section 20 of the corresponding arm 12. Further, the guide 40 has a rearward opening 42 at a rearward end facing the rearward end 14 of the instrument 10 and a forward opening 44 at a forward end facing the working end 22 and jaw 30 of the corresponding arm 12. In the illustrated example, the rearward opening is oriented perpendicular to the guide 40 centerline of the tube but the forward opening 44 is obliquely oriented relative to the centerline. As will be appreciated, the oblique orientation of the forward opening 44 facilitates threading the electrode leads (not shown) through the guide 40.

Each arm 12 has a seat or grip 50 including a series of parallel grooves to facilitate a user reliably grasping and manipulating the forceps 10. The seat 50 is provided on the outer face 26 along the rearward section 16 of each arm 12. As will be appreciated, the arms 12 are resiliently flexible. When a user grips the seats 50 between a thumb and index finger and squeezes the seats toward each other, the gap distance 32 between opposing clamping faces 34 of the jaws 30 closes. When the user releases pressure on the seats 50, the gap distance 32 between the jaws 40 widens. Accordingly, the forceps 10 are adapted for selectively grasping items between the jaws 30. In some examples, the arms 12 are sufficiently flexible that when a user applies a typical forceps closing pressure on the seats 50, the gap distance 32 closes to about 8 mm enabling the forceps 10 to grip paddle electrodes from several commercial brands, which typically have widths of about 8-12 mm. It is envisioned that the flexibility may be increased or decreased, and the lengths of the arms may be adjusted to accommodate other brands and other objects. In the illustrated examples, the arms 12 are sufficiently resilient to return to an initial, undeformed shape having a gap distance 32 of about 30 mm. Other undeformed gap distances are envisioned.

As will be appreciated, the jaws 30 are separated by a gap distance 32 that is larger than a distance between the opposite side edges of the paddle electrode when the arms is not flexed. The arms 12 are capable of being flexed to reduce the gap distance 32 separating the jaws 30 sufficiently to engage the jaws with the side edges of the paddle electrode to grip the paddle electrode. Further, the arms 12 are sufficiently resilient to return to a released configuration in which the gap distance 32 is larger than the distance between the opposite side edges of the paddle electrode.

The instrument 10 described above may be manufactured using common manufacturing techniques used to make surgical forceps. Further, the materials used to make the instrument 10 are similar to those used to make conventional bayonet forceps. As the materials and manufacturing techniques are conventional, they will not be described further.

One technique for positioning a paddle electrode using the specialized bayonet forceps 10 described above consists of making an incision in the skin overlaying the patient's spine. The incision length should be minimized (e.g., about 4 cm) at a level corresponding to the intended placement or target site (i.e., usually between vertebra T9 and T11). The practitioner then cuts tissues below the skin, including muscles surrounding and attached to the spine to provide a generally straight opening exposing the bilateral laminae (i.e., layered ligaments bridging the vertebrae) and the spinous process (i.e., boney vertebral protrusions). Surgical retractors (e.g., a cerebellar retractors) are positioned to maintain the opening and expose the spinal canal. The practitioner confirms that the location of the incision corresponds to the desired target using fluoroscopy before removing the spinous process (e.g., using a Leksell rongeur). After removing the vertebral protrusions, the practitioner cuts the bilateral laminae (e.g., using a drill) and separates the ligamentum flavum (i.e., a ligament extending between the vertebrae adjacent the spinal canal) from the dura. The final laminectomy (i.e., opening into the spinal canal) has a craniocaudal diameter (i.e., extending lengthwise relative to the spine) of about 20 mm and a width of 10 mm.

The practitioner grips the specialized bayonet forceps 10 by the seats and moves the instrument so the jaws 30 are adjacent to the side edges of the paddle electrode so the edges align with a selected groove in the jaw. The practitioner squeezes the seats, bringing the jaws 30 together on the paddle electrode at a position about midway along the electrode between its leading and trailing edges. When the bayonet forceps 10 are closed by firmly squeezing the seats 50, the jaws 30 of the forceps close so the distal-most grooves 36 in the jaws 30 engage the side edges of the paddle electrode to firmly hold the paddle electrode between the jaws. The leads of the paddle electrodes are inserted into the obliquely oriented forward opening 44 through the guide 40 and out from the rearward opening 42. As the leads are gently pulled, the paddle electrode flexes so craniocaudal diameter is reduced by at least 30%. With the paddle electrode flexed, the paddle electrode is gently inserted underneath the cranial lamina and spinous process until the working ends 22 of the specialized forceps 10 contacts the cranial bony edge. Once in this position, the grip pressure is relaxed allowing the jaws 30 to open releasing the paddle electrode from the grooves 36. The leads are removed from the grooves by pulling them. This maneuver may be repeated if the paddle electrode is desired to be advanced upward relative to the patient. The leading edge of the paddle electrode can be grasped between the grooves by applying closing pressure at the forceps seats 50. Usually, it is not necessary to rethread the leads through the guides 40 after the paddle electrode is inserted part way and the remaining length is no more than the length of the incision.

As skilled observers will note, several advantages are achieved by using the instrument 10 to insert a spinal stimulation paddle electrode. In particular, the instrument 10 that ensures the practitioner is able to firmly grip the paddle electrode by its side edges and guide its leads when flexing the paddle electrode to guide the electrode into place through a minimally invasive incision.

When introducing elements in this description and the claims, the articles “a”, “an”, “the”, and “said” are intended to indicate one or more of the elements. The terms “comprising”, “including”, and “having” are intended to be inclusive and indicate there may be additional elements other than the listed elements.

As those skilled in the art could make various changes to the above constructions, products, and methods without departing from the intended scope of the description, all matter in the above description and accompanying drawings should be interpreted as illustrative and not in a limiting sense. The patentable scope of the disclosure is defined by the claims, and can include other constructions and methods that would occur to those skilled in the art. Such other constructions are intended to be within the scope of the claims if the structural elements of the constructions do not differ from the literal language of the claims, or if the constructions include equivalent structural elements having insubstantial differences from the literal languages of the claims.

To the extent that the specification, including the claims and accompanying drawings, discloses any additional subject matter that is not within the scope of the claims below, the disclosures are not dedicated to the public and the right to file one or more applications to claims such additional disclosures is reserved.

1. A surgical instrument for positioning a paddle electrode having opposite side edges extending between a leading edge and a trailing edge opposite the leading edge, the paddle electrode having leads extending from the trailing edge, said instrument comprising:
a pair of flexibly resilient arms operatively coupled at a user end of each arm of said pair of arms, each arm of said pair of arms extending longitudinally along a rearward section from the corresponding user end to an intermediate section extending obliquely upward relative to the rearward section to a forward section extending obliquely downward relative to the intermediate section toward a working end opposite the corresponding user end, each arm of said pair of arms having a cooperative face facing the other arm of said pair of arms and an outer face facing opposite said cooperative face;
a jaw formed adjacent to the working end of each arm of said pair of arms adapted to engage a selected side edge of the paddle electrode when gripping the paddle electrode; and
a guide located along the forward section of each arm of the pair of arms adapted to receive a selected lead of the leads of the paddle electrode when the jaws of said arms of said pair of arms are gripping the paddle electrode; wherein:
the jaws of said pair of arms are separated by a gap that is larger than a distance between the opposite side edges of the paddle electrode when said pair of arms is unflexed;
said pair of arms is capable of being flexed to reduce the gap separating the jaws of said pair of arms sufficiently to engage the jaws of said pair of arms with the side edges of the paddle electrode to grip the paddle electrode; and
said pair of arms is sufficiently resilient to return said pair of arms to a released configuration in which the gap separating the jaws of said pair of arms is larger than the distance between the opposite side edges of the paddle electrode.

2. A surgical instrument as set forth in claim 1, wherein each jaw formed adjacent to the working end of each arm of said pair of arms is offset from the working end of the corresponding arm of said pair of arms.

3. A surgical instrument as set forth in claim 2, wherein the gap by which the jaws of said pair of arms are separated is larger than a space between the working ends of the arms of said pair of arms.

4. A surgical instrument as set forth in claim 1, wherein each jaw has a groove facing the jaw of the other arm of said pair of arms.

5. A surgical instrument as set forth in claim 4, wherein each groove has a groove width is in a width range of about 3 mm to about 4 mm.

6. A surgical instrument as set forth in claim 4, wherein:
each said groove is a first groove; and
each jaw has a plurality of parallel grooves including the first groove facing the jaw of the other arm of said pair of arms.

7. A surgical instrument as set forth in claim 1, wherein each jaw has a concave face facing the jaw of the other arm of said pair of arms.

8. A surgical instrument as set forth in claim 1, further comprising a seat formed on the outer face of each arm of said pair of arms adapted to grasp and manipulate the instrument.

9. A surgical instrument as set forth in claim 8, wherein the seat formed on the outer face of each arm of said pair of arms is located on the intermediate section each arm of said pair of arms.

10. A surgical instrument as set forth in claim 9, wherein said pair of arms are capable of being flexed using a conventional pressure applied to the seats to reduce the gap separating the jaws of said pair of arms to a gap width in a gap width range of about 8 mm to about 12 mm.

11. A surgical instrument as set forth in claim 1, wherein each guide comprises a hollow tube mounted on the forward section of the corresponding arm of said pair of arms.

12. A surgical instrument as set forth in claim 11, wherein the hollow tube of each guide has a minimum interior width of about 3 mm.

13. A surgical instrument as set forth in claim 11, wherein each hollow tube is cylindrical having a centerline extending parallel to the forward section of the corresponding arm of said pair of arms.

14. A surgical instrument as set forth in claim 13, wherein each hollow tube has a rearward facing opening that is obliquely oriented relative to the corresponding centerline.

15. A surgical instrument for positioning a paddle electrode having opposite side edges extending between a leading edge and a trailing edge opposite the leading edge, the paddle electrode having leads extending from the trailing edge, said instrument comprising:
a pair of flexibly resilient arms operatively coupled at a user end of each arm of said pair of arms, each arm of said pair of arms extending to a working end opposite the corresponding user end, each arm of said pair of arms having a cooperative face facing the other arm of said pair of arms and an outer face facing opposite said cooperative face;
a jaw formed at and offset from the working end of each arm of said pair of arms, each said jaw having a concave face facing the jaw of the other arm of said pair of arms and a groove extending across said concave face, the width of said groove being adapted to receive a paddle electrode side edge; and
a guide located along each arm of the pair of arms adapted to receive a selected lead of the leads of the paddle electrode when the jaws of said arms of said pair of arms are gripping the paddle electrode; wherein:
the jaws of said pair of arms are separated by a gap that is larger than a distance between the opposite side edges of the paddle electrode when said pair of arms is unflexed;
said pair of arms is capable of being flexed to reduce the gap separating the jaws of said pair of arms sufficiently to engage the jaws of said pair of arms with the side edges of the paddle electrode to grip the paddle electrode; and
said pair of arms is sufficiently resilient to return said pair of arms to a released configuration in which the gap separating the jaws of said pair of arms is larger than the distance between the opposite side edges of the paddle electrode.

16. A surgical instrument as set forth in claim 15, wherein the width of each groove is in a width range of about 3 mm to about 4 mm.

17. A surgical instrument as set forth in claim 4, wherein:
each said groove is a first groove; and
each jaw has a plurality of parallel grooves including said first groove extending across the corresponding concave face.

18. A surgical instrument for positioning a paddle electrode having opposite side edges extending between a leading edge and a trailing edge opposite the leading edge, the paddle electrode having leads extending from the trailing edge, said instrument comprising:
a pair of flexibly resilient arms operatively coupled at a user end of each arm of said pair of arms, each arm of said pair of arms extending to a working end opposite the corresponding user end, each arm of said pair of arms having a cooperative face facing the other arm of said pair of arms and an outer face facing opposite said cooperative face;
a jaw formed at and offset from the working end of each arm of said pair of arms, each said jaw having a concave face facing the jaw of the other arm of said pair of arms; and
a guide comprising a hollow cylindrical tube mounted on each arm of said pair of arms adjacent to the working end of the corresponding arm of said pair of arms, said hollow cylindrical tube extending along a centerline of the corresponding arm of said pair of arms and having an interior diameter sized to receive at least one lead of said paddle electrode leads; wherein:
the jaws of said pair of arms are separated by a gap that is larger than a distance between the opposite side edges of the paddle electrode when said pair of arms is unflexed;
said pair of arms is capable of being flexed to reduce the gap separating the jaws of said pair of arms sufficiently to engage the jaws of said pair of arms with the side edges of the paddle electrode to grip the paddle electrode; and
said pair of arms is sufficiently resilient to return said pair of arms to a released configuration in which the gap separating the jaws of said pair of arms is larger than the distance between the opposite side edges of the paddle electrode.

19. A surgical instrument as set forth in claim 18, wherein the interior diameter of each hollow cylindrical tube is about 3 mm.

20. A surgical instrument as set forth in claim 18, wherein each hollow cylindrical tube has a rearward facing opening that is obliquely oriented relative to the centerline of the corresponding arm.
